(19) United States
(12) Patent Application Publication
(10) Pub. No.: US 2021/0307969 A1
(43) Pub. Date: Oct. 7, 2021

(54) AN ARRAY OF PANT-TYPE ARTICLES, AND A METHOD OF PRODUCING PANT-TYPE ARTICLES IN AN ARRAY OF PANT-TYPE ARTICLES

(71) Applicant: Essity Hygiene and Health Aktiebolag, Göteborg (SE)

(72) Inventor: Karin LJUNGBERG, Göteborg (SE)

(73) Assignee: Essity Hygiene and Health Aktiebolag, Göteborg (SE)

(21) Appl. No.: 17/266,161

(22) PCT Filed: Aug. 6, 2018
(86) PCT No.: PCT/SE2018/050792

§ 371 Date: Feb. 5, 2021

(51) Int. Cl. A61F 13/15; A61F 13/496

(52) U.S. Cl. CPC A61F 13/15699; A61F 13/15804; A61F 13/15747; A61F 13/496

(57) ABSTRACT

A method for producing an array of disposable pant-type articles, the array including at least a first and a second pant-type article having different sizes. The method includes a number of different processes corresponding to the number of pant-type articles in the array wherein all processes involve forming a stretchbonded laminate web comprising a continuous elastic web and a non-elastic or less elastic web, forming a composite web of precursor articles including the stretchbonded laminate web and separating individual pant-type articles from the composite web by severing the composite web at side joins formed between the precursor articles. In the different processes, the continuous elastic web differs between the different processes with regard to elastic properties while a pitch length of the stretchbonded elastic laminate web is the same in all continuous processes of the method. Also, an array of pant-type articles which may be produced by the method.

### TECHNICAL FIELD

The disclosure relates to an array of pant-type articles differing in size. The pant-type articles may be disposable pant-type articles, and may be absorbent or non-absorbent pant-type articles. The pant-type articles of the array of pant-type articles may be produced according to a method for producing pant-type articles for an array of pant-type articles, as disclosed herein. The method comprises a first continuous process for producing a first pant-type article having a first size and a second continuous process for producing a second pant-type article having a second size.

### BACKGROUND

In the field of pant-type articles, such as disposable absorbent pant-type articles, there is a general desire to provide pant-type articles with increased comfort and fit as well as discrete underwear-like visual appearance. Due to anatomical and size differences between different users, such as difference in anatomy between men and women, difference in body shape between users of different girth, etc., a wide range of absorbent articles of different sizes needs to be provided. To date, the production of differently sized pant-type articles within an array of articles has required the use of different manufacturing machines, which leads to increased manufacturing costs. Thus, there is a continuing need for improvements in particular with regard to cost efficiency in the manufacturing of pant-type articles of different sizes. There is also a need for an array of pant-type articles adapted to a wide range of users with regard to comfort, fit and discrete underwear-like visual appearance which array of pant-type articles can be produced in a cost efficient manner.

### SUMMARY

It is an object of the present disclosure to provide an array of pant-type articles, such as disposable pant-type articles, which can be produced in a cost-efficient manner. It is also an aim to provide an efficient method of producing pant-type articles which form part of an array of pant-type articles. These and other objects are achieved at least in part by the features of the independent claims. Variations of the disclosure are set out in the dependent claims.

Disclosed herein is an array of pant-type articles, such as an array of absorbent pant-type articles including an absorbent core, the array of pant-type articles including a first pant-type article and a second pant-type article, each first pant-type article and second pant-type article comprising a chassis, the chassis comprising a front panel, a back panel and a crotch portion between the front and back panels, the front panel having a waist edge and a pair of side edges, and the back panel having a waist edge and a pair of side edges, the side edges of the front and back panels being joined in a first and a second side seam, whereby the pant-type article has a waist opening and a first and a second leg opening. Each of the first pant-type article and the second pant-type article comprises a stretchbonded elastic laminate web which extends from the first side seam to the second side seam over all or a part of the front panel and/or of the back panel of the chassis, wherein a body portion circumference at 10 Newton of the first pant-type article in the array is at least 20% larger than a body portion circumference of the second pant-type article in the array at 10 Newton, a body portion circumference at 30 Newton of the first pant-type article in the array is at least 20% larger or at least 25% larger than a body portion circumference of the second pant-type article in the array at 30 Newton, and a body portion circumference at 90 Newton of the first pant-type article in the array is less than 10% larger than a body portion circumference of the second pant-type article in the array at 90 Newton as measured by the body portion circumference test as disclosed herein.

The stretchbonded elastic laminate web may constitute a sole component of said chassis in 20%-100%, preferably 25%-100%, more preferably 30%-100% and most preferably 40%-100% of a total surface area of said pant-type article, as seen in a flat state.

As set out herein, the stretchbonded elastic laminate web may be applied in one or both of the front panel and the back panel and may also be applied in the crotch portion.

Disclosed herein is also a method for producing pant-type articles in an array of pant-type articles, the array of pant-type articles comprising a first pant-type article and a second pant-type article, the first and second pant-type articles differing in size. The method comprises a first continuous process for producing the first pant-type article and a second continuous process for producing the second pant-type article, each of the first and second continuous processes including the steps of:
providing a continuous elastic web and moving the continuous elastic web in a machine direction, the continuous elastic web being elastically stretchable in the machine direction;
stretching the continuous elastic web in the machine direction;
bonding the stretched continuous elastic web while held in the stretched state to a non-elastic or less elastic web to form a stretchbonded elastic laminate web;
forming a composite web of article components, the composite web constituting a continuous web having leg openings arranged therein and consisting of interconnected precursor articles, the composite web comprising the stretchbonded elastic laminate web as a chassis component;
folding the composite web along a fold line extending centrally along the composite web in the machine direction;
forming side joins in the folded composite web; and
separating individual pant-type articles from the composite web by severing the composite web at the side joins. As set out herein, the continuous elastic web in the first continuous process is different from the continuous elastic web in the second continuous process with regard to elastic properties while a pitch length of the stretchbonded elastic laminate web is the same in the first continuous process and said second continuous process.

Forming the composite web of article components may comprise:
moving the stretchbonded elastic laminate web as a first continuous web in the machine direction alongside a second continuous web arranged at a distance from the first continuous web in a cross machine direction perpendicular to the machine direction with a gap between the first continuous web and the second continuous web;
connecting the first continuous web with the second continuous web by attaching a crotch material to the first continuous web and to the second continuous web, the crotch material bridging the gap between the first continuous web and the second continuous web, wherein the second continuous web is a non-elastic or elastic web.

A process of this kind produces a two-part chassis wherein the first and second continuous webs form front and back panels in the pant-type article and the interconnecting crotch material forms a crotch panel between the front and back panels. The crotch material may be discrete piece of material which are attached intermittently and spaced apart in the machine direction. Leg openings are automatically formed in the spaces between the intermittently applied crotch material pieces. If desired, the shape of the leg openings may be enhanced by cutting.

The second continuous web may be a non-elastic web, but is preferably an elastic web having the same pitch length or a greater pitch length than the stretchbonded elastic laminate web forming the first continuous web. At the same pitch-length, the second continuous web may be made from the same materials as the first continuous web or may be made from materials differing from those of the first continuous web. The materials may differ in composition, and/or in physical properties such as basis weight, thickness, permeability, etc. Non-woven webs may differ in kind such as being a bonded-carded web, a spunbond web, a meltblown web, etc. The second continuous web may have a different elastic layer from that of the first continuous web, such as an elastic layer having different elastic properties and/or may include one or more non-elastic layer or layers different from the first continuous web such as different non-woven layers.

Alternatively, the crotch material is a continuous web, wherein leg openings are cut in the web after application of the crotch material.

When the pant-type article is an absorbent article, an absorbent core is applied at least in the portion of each precursor article which will form the crotch panel in the fully assembled article. Preferably, the absorbent core is applied with a portion of the core overlapping with one or both of the front and back panels. As set out herein, the absorbent core may be applied as a separately produced core insert. In such case, the core insert may constitute an intermittently applied crotch material.

Alternatively, the step of forming the composite web of article components may comprise:
moving the stretchbonded elastic laminate web in the machine direction;
intermittently cutting out leg openings in the stretchbonded elastic laminate web.

A process of this kind produces a unitary chassis as set out herein.

When the pant-type article is an absorbent article, an absorbent core is applied at least in the portion of each precursor article which will form the crotch portion or crotch panel in the fully assembled article. Preferably, the absorbent core is applied with a portion of the core overlapping with one or both of the front and back portions or front and back panels. As set out herein, the absorbent core may be applied as a separately produced core insert.

In a composite web comprising absorbent cores, the leg openings are formed between the absorbent cores. As is common in the art, each leg opening in the composite web forms a leg edge of two consecutive precursor articles in the moving composite web.

In addition to the first and second continuous processes, the method as disclosed herein may include one or more further continuous processes such that the number of continuous processes in the method is 2+n, corresponding to 2+n different pant-type articles in the array of pant-type articles, wherein n is an integer≠0. Each of the one or more further continuous processes includes the process steps of the first and second continuous processes and produces a pant-type article which is different in size from every other pant-type article produced by another of the 2+n continuous processes comprised in the method, wherein the continuous elastic web in every further continuous process is different from the continuous elastic web in every other continuous process in the method with regard to elastic properties and wherein a pitch-length of the precursor articles is the same in the 2+n continuous processes.

Each additional continuous process produces a pant-type article which differs in size from any other pant-type article produced according to another continuous process of the method for producing pant-type articles included in an array of pant-type articles, as set out herein.

As set out herein, the integer n may be from 1 to 5, such as from 1 to 3 or from 1 to 2, corresponding to 3 to 7, 3 to 5 and 3 to 4 different pant-type articles in the array of pant-type articles.

By selecting different continuous elastic webs with different elastic properties for each process of the method for producing an array of pant-type articles, pant-type articles of different sizes can be produced using elastic webs having the same pitch length in the machine. Thereby, the extension in the machine direction of the precursor articles in one of the processes of the method as disclosed herein will be the same or substantially the same as the extension in the machine direction of the precursor articles of any other process comprised in the method as disclosed herein. This allows the same machine parts and the same settings or substantially the same settings to be used for the first, second and any further continuous process of the method which means that the production of different pant-type articles being part of an array of pant-type articles of different sizes may be produced in a highly simplified and cost-efficient manner with the same type of production equipment. According to the method as disclosed herein, it is even possible to run the first continuous process, the second continuous process and any further continuous process on a same manufacturing machine without having to change the machine settings or investing in and using different machine parts for different sizes of the produced pant-type articles in the array of pant-type articles.

In the method as disclosed herein, each continuous elastic web used in one of the continuous processes of the method may differ from every other continuous elastic web used in another of the continuous processes in one or more aspects selected from:
physical structure
polymer composition;
basis weight;
thickness
permanent set
degree of orientation in machine direction of the elastic web/film.

The physical structure of the continuous elastic web may be an elastic film, an elastic fibrous web or an elastic laminate of two or more layers, such as a film/nonwoven laminate. Elastic film/nonwoven laminates include stretchbonded laminates of an elastic web and at least one non-elastic or less elastic web as well as incrementally activated laminates of an elastic web and at least one non-elastic or less elastic web. The elastic film/nonwoven laminates may include an elastic film and a non-elastic or less elastic nonwoven web which is bonded to one or both surfaces of the elastic film. Bonding may be carried out by ultrasonic welding, thermobonding, extrusion bonding, or by using an adhesive. In a multi-layer laminate, such as a nonwoven-film-nonwoven laminate, the different nonwoven layers may be bonded to the film using different bonding techniques.

As known in the art, an elastic film may have a layered structure with a central core layer and a skin layer on one or both surfaces of the central core layer. At the same polymer composition in the core layer and skin layer or skin layers and/or the same thickness of the elastic film, the elastic films used in the different processes of the method as disclosed herein may differ in a relation of a thickness of a skin layer or skin layers to the thickness of the core layer.

The elastic webs as disclosed herein are elastic in at least the machine direction and may be elastic also in the cross machine direction. In the finished pant-type article, the machine direction corresponds to the width direction of the article while the cross machine direction corresponds to the length direction of the article.

In the method as disclosed herein, the elastic web may be stretched in the machine direction by at least 90% of an initial non-stretched length during stretch-bonding, such as by up to 150% of the initial non-stretched length, preferably by up to 300% of the initial non-stretched length and most preferably by up to 500% of the initial non-stretched length.

In the processes of the method as disclosed herein, the stretched elastic web may be bonded to the at least one non-elastic or less elastic web by means of adhesive that is applied to one or both webs by means of coating, spraying, extrusion or meltblowing, or that the stretched elastic web is bonded to the non-elastic or less elastic web by means of ultrasonic welding or thermobonding.

In the pant-type articles as disclosed herein, the article components may include one or more of:
an absorbent core or absorbent insert
a waist elastic member;
a leg elastic member;
an inner liner;
an acquisition layer;
a barrier layer;
a barrier cuff; and
a fastener element.

In the method as disclosed herein, the continuous elastic web may be an elastic film, preferably a breathable elastic film, such as a perforated elastic film or a microporous elastic film. The non-elastic or less elastic web may be a nonwoven web, such as a carded bonded nonwoven web, a spunbond nonwoven web or a creped nonwoven web.

Disclosed herein is also an array of pant-type articles including a first pant-type article and a second pant-type article, wherein the array of pant-type articles may be produced according to the method as disclosed herein and wherein the first pant-type article differs in size from that of the second pant-type article. The array of pant-type articles as disclosed herein may comprise 2+n different kinds of pant-type articles, wherein n is an integer≠0, such as 1 to 5 or 1 to 3 or 1 to 2.

The pant-type articles as disclosed herein are preferably disposable absorbent articles, i.e. articles including an absorbent core.

As set out herein, the array of pant-type articles as disclosed herein comprises at least a first and a second article. The circumference at 10 Newton of the first article may be at least 20% larger than the circumference of the second article at 10 Newton when measured according to the test method disclosed herein. Accordingly, the first article has a larger size than the second article.

Furthermore, the circumference at 30 N for the first article may be at least 20% larger or at least 25% larger than the circumference at 30 N for the second article when measured according to the test method disclosed herein. Accordingly, the first article has a larger size than the second article.

Furthermore, the circumference at 90 Newton for the first article may be less than 10% larger, such as less than 5% larger than the circumference at 90 Newton for the second article when measured according to the test method disclosed herein. This measurement shows that the materials in the different pant-type articles in an array of articles produced according to the method as disclosed herein, have been produced with elastic web materials having the same pitch length or substantially the same pitch length in the machine direction, as a maximum extension of the waist circumference is in the same order for the first and the second pant-type article.

In this context, an elastic material is defined as a material having a permanent elongation after relaxation of less than 10% after the material has been subjected to an elongation of 30% in the elasticity test specified in the description.

A non-elastic material is a material that does not fall within the definition of an elastic material. Accordingly, a non-elastic material as used herein is a material that may be stretchable or non-stretchable. In the case of a stretchable non-elastic material, the material has a permanent elongation after stretching and relaxation of more than 10% after having been subjected to an elongation of 30% as determined according to the elasticity test. The non-elastic or less elastic materials used in the stretchbonded laminates as disclosed herein have a permanent elongation after stretching and relaxation of less than 10% after having been subjected to an elongation of 30% as determined according to the elasticity test.

The pitch-length of the stretchbonded elastic laminate web is measured in the machine direction and is the length of the extended stretchbonded laminate web of a single precursor pant-type article in the machine direction. The pant-type articles as disclosed herein are produced with their length direction parallel with the cross machine direction and with their width direction parallel with the machine direction.

As defined herein, an array is a set of articles or packages which are intended to be marketed and/or displayed together. An array of pant-type articles is a set of pant-type articles which are related to each other in kind, by being similar in one or more aspects such as construction, appearance and function, but which differ from each other e.g. by having different size, being designed for a particular gender, having different absorption capacity, etc. The array of pant-type articles as disclosed herein comprises at least two pant-type articles having the same or similar construction but differing in size.

By an “absorbent article” as used herein is implied an article that absorbs or is adapted to absorb bodily fluids, such as urine, blood and loose fecal matter.

The absorbent article comprises an absorbent core which may comprise any conventional material suitable for absorbing discharged bodily wastes, such as cellulosic fluff pulp, tissue layers, highly absorbent polymers (super absorbents), absorbent foam materials including hydrogel-foam material, absorbent nonwoven materials, or the like. The absorbent core can comprise non-absorbent components such as stiffening elements, shaping elements, binders, etc. Various types of liquid-receiving and liquid distribution elements can also be included in the core.

The absorbent article may further be provided with a liquid permeable inner liner, also referred to herein as a topsheet covering the absorbent core on the side of the core which is intended to face the skin of a user, and a liquid impermeable backsheet covering the absorbent core on the side of the core which is intended to face the garment of a user. The liquid permeable topsheet may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet.

The liquid impermeable backsheet may consist of a thin plastic film, e. g. a polyethylene or polypropylene film, a nonwoven material coated with a liquid impervious material, a hydrophobic nonwoven material which resists liquid penetration or laminates of plastic film and nonwoven. The backsheet material may be breathable so as to allow vapour to escape from the absorbent body, while still preventing liquids from passing through the backsheet material.

The topsheet and backsheet may be connected to each other for example by adhesive bonding, gluing or welding by heat or ultrasonically. The topsheet and/or the backsheet may further be attached to the absorbent core by any method known in the art, such as adhesive, heat-bonding, welding, needling, etc.

The absorbent core may be applied to the pant-type absorbent article as disclosed herein in the form of a separately produced absorbent insert. The absorbent insert may comprise an absorbent core enclosed between a topsheet and a backsheet. The topsheet and the backsheet may extend outwardly beyond the area of the absorbent core and may be attached to each other outside the edges of the absorbent core. Alternatively, the backsheet may be applied to the garment-facing side of the absorbent core, whereafter the core and the backsheet may be wrapped in a topsheet material. The absorbent insert may comprise further components such as a fluid acquisition layer, a liquid distribution layer, shape stabilizing components, elastic elements, barrier elements, etc. as known in the art.

The pant-type articles as disclosed herein may be of the kind having a unitary chassis or may be of the kind having a two-part chassis.

A unitary chassis extends without interruption in a length direction of the pant-type article from a first waist opening edge through a front portion of the article, a crotch portion of the article and a rear portion of the article to a second waist opening edge. The unitary chassis includes a front panel, a back panel and an interconnecting crotch panel and is fully or partly formed from a stretch bonded laminate as produced in the continuous processes of the method for forming an array of pant-type articles as disclosed herein. At least one layer of the unitary chassis extends continuously from the first waist opening edge to the second waist opening edge.

A two-part chassis may be produced by first manufacturing two parallel continuous strips of web material which form front and rear body panels in the assembled pant-type article. The strips are fed parallel to each other with a gap between the strips and are intermittently connected by application of a crotch material which bridges the gap between the strips of laminated elastic web material. The crotch material may be an absorbent insert, as disclosed herein. At least one of the strips of web material is an elastic web material, preferably a stretchbonded laminated elastic web material as disclosed herein.

The stretch-bonded laminates as disclosed herein, may comprise nonwoven material layers or webs such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibres are for instance cellulosic fibres such as pulp fibers, cotton fibers, flax, hemp, etc.

The pant-type articles of the arrays of pant-type articles as disclosed herein may have an elastic waist feature along all or part of the waist opening. An elastic waist feature may be formed by one or more elastic elements in the composite web of article components. The elastic element or elements are attached along one or both of the outer edges of the composite web of article components, which outer edges are placed at the waist opening in the assembled pant-type article. The elastic waist element or elements may be incorporated in the stretchbonded elastic laminate formed in the processes in the method as disclosed herein or may be applied after forming the stretchbonded elastic laminate. Furthermore, the elastic element or elements may be placed inside a fold in the stretchbonded elastic laminate web such that the elastic element or elements are enclosed in the fold.

Alternatively, the elastic elements can be covered by or attached to another web of material being a part of the composite web of article components, such as an inner or outer nonwoven layer, or may be provided in the form of a separately produced waistband which is attached as a component of the composite web of article components. The waist elastic elements are preferably attached in a tensioned state so as to contribute to the elastic gathering force at the waist edges.

By the term “elastic element” is implied an elongated elastic element such as an elastic strand, an elastic thread or an elastic band. The elastic material in the elastic element may be any suitable elastic material such as natural or synthetic rubber, thermoplastic elastomers, such as thermoplastic polyurethane or styrene block co-polymers or elastane, also referred as to spandex (polyurethane-polyurea copolymer). The elastic elements may be of the elastane type that is available under the trade name “LYCRA”, but any suitable elastic thread may be used. The threads may have a linear mass density, dtex, of about 80-1200 dtex. Elastic elements may be used in the method as disclosed herein for producing elastic features such as waist elastic, leg elastic and elastic barriers.

The side joins connect the front portion of the pant-type article to the rear portion, whereby the precursor pant-type articles are formed into a pant having a waist opening and leg openings. Usually, the side joins are intended to be arranged at the user's hips during use of the pant-type article, but it is also known to arrange side joins more to the front of the article. The side joins are preferably designed so that they can withstand the tensile forces which arise when the pant-type article is being put on and is being worn, but such that they can be torn apart or opened in a controlled manner when the pant-type article is taken off or to check if the article needs changing. In the latter instance, the side joins are preferably reclosable joins, as known in the art.

The side joins may be produced in the processes of the method as disclosed herein as a single broad join between the individual precursor articles, which join is divided in two parts when separating the finished pant-type articles from the continuous web of precursor articles. Alternatively, two separate side joins may be formed side by side between the individual precursor articles whereafter the continuous web of precursor pant-type articles is severed between the two side joins.

The non-elastic or less elastic web or webs may comprise thermoplastic material. The non-elastic or less elastic web or webs webs will typically be incorporated in joins and seams in the pant-type article. It may be generally preferred that the non-elastic or less elastic web or webs are fibrous, nonwoven webs. It is highly desirable that the nonwoven webs be weldable by heat or by ultrasonic welding processes. Examples of suitable polymers for use in the nonwoven webs are polyethylene polypropylene and other polyolefin homopolymers and copolymers and polyesters. The weldable nonwoven webs have a high content of thermoplastic component and preferably contain at least 50% thermoplastic fibres and more preferably at least 80% thermoplastic fibres.

The elastic web is preferably a breathable elastic film such as an apertured or microporous elastic film. The elastic film may have a basis weight of between 20 and 80 g/m², preferably between 20 and 60 g/m². The elastic web may be of any suitable elastic polymer, natural or synthetic. Some examples of useful materials for the elastic web are low crystallinity polyethylenes, metallocene-catalyzed low crystallinity polyethylenes, ethylene vinyl acetate copolymers (EVA), polyurethane, polyisoprene, butadiene-styrene copolymers, styrene block copolymers, such as styrene/isoprene/styrene (SIS), styrene/butadiene/styrene (SBS), or styrene/ethylene-butadiene/styrene block copolymer. Blends of these polymers may also be used as well as other modifying elastomeric or non-elastomeric materials. The total basis weight of the stretchbonded elastic laminate web is preferably between 40 and 100 g/m², more preferably not more than 90 g/m².

### BRIEF DESCRIPTION OF THE DRAWINGS

The present invention will be further explained hereinafter by means of non-limiting examples and with reference to the appended drawings wherein:

FIG. 1a shows a front view of an example pant-type article with a two-part chassis;

FIG. 1b shows a front view of an example pant-type article with a unitary chassis;

FIG. 2 shows a schematic representation of a process for producing a stretchbonded laminate;

FIG. 3 shows a schematic representation of a process for producing a pant-type article with a two-part chassis;

FIG. 4 shows a schematic representation of a process for producing a pant-type article with a unitary chassis;

FIG. 5 shows the test equipment used for determining the circumference of the waist portion in an extended state and in a relaxed state of the pant-type article, as well as for determining the circumference of the body portion in the extended state of the pant-type article; and

FIG. 6 shows a test diagram with a comparison between two different tested pant-type articles.

### DETAILED DESCRIPTION

It is to be understood that the drawings are schematic and that individual components, such as layers of material are not necessarily drawn to scale. The disposable pant-type absorbent article shown in the figures is provided as an example only and should not be considered limiting to the invention as disclosed herein. As set out herein, the method of producing different sized pant type articles is applicable also to non-absorbent articles and reusable pant-type articles or articles which can be laundered and reused a limited number of times. Reusable articles which may withstand laundering may be non-absorbent pant-type articles e.g. pant-type articles which are adapted for use together sin an absorbent insert.

Furthermore, the construction of the pant-type articles may be different from those shown in connection with the figures. As set out herein, an alternative construction of a unitary chassis may comprise a single continuous outer cover sheet, and/or a continuous inner liner and an elastic panel webs joined to one or both of the front and back part of the chassis to form an elastic front or back panel, respectively.

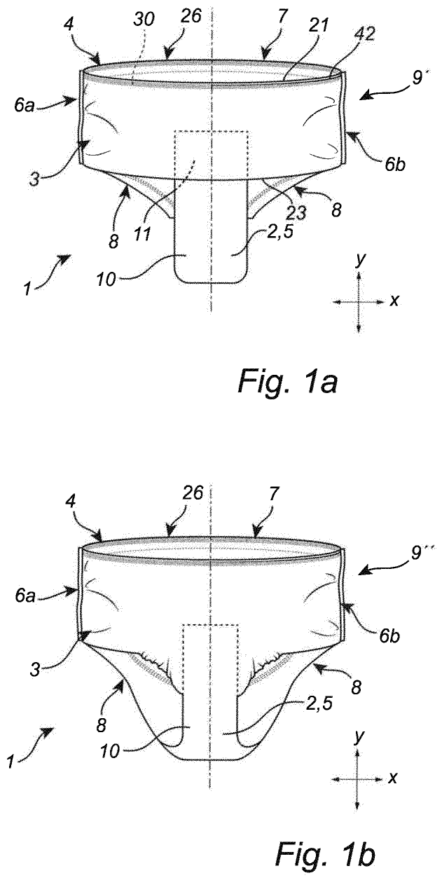

The pant-type articles shown in the figures are simplified articles, and it should be understood that they may be enhanced with elastic elements arranged at the leg openings on one or more of the front panel, the back panel and in the crotch portion. Further elastification may be provided e.g. in the form of barrier cuffs. It is also to be understood that the waist elastic disclosed in connection with FIGS. 1a and 1b is optional or that it may be substituted by any other type of elastic waist feature as known in the art. As set out herein, the side seams may be reclosable side seams, and the pant-type article may be provided with fastener elements to provide reclosability of the side seams.

The pant-type absorbent article 1 shown in FIG. 1a has a longitudinal direction Y and a transverse direction X and comprises a two-piece chassis 9′ having a front panel 3, a back panel 4 and a core insert 2 located mainly in a crotch portion 10 of the pant-type absorbent article 1. The core insert 2 bridges a gap between the front and back panels 3, 4 and extends with end portions 11 in over the front and back panels 3,4 and is connected at the end portions 11 to an interior side of the front and back panels 3, 4. As set out herein, the core insert 2 is a separately produced component which comprises an absorbent core 5 for absorbing body fluid. The front panel 3 has a waist edge 21 and a pair of side edges, and the back panel 4 has a waist edge 26 and a pair of side edges. The side edges of the front and back panels 3,4 are joined in side seams 6a, 6b, thereby forming the pant-type article 1 having a waist opening 7 and two leg openings 8.

The pant-type absorbent article 1 which is schematically shown in FIG. 1a is illustrated in an extended state and is fully assembled and ready-to-use. The pant-type absorbent article 1 may be a pant diaper, a sanitary pant or an incontinence pant and may be adapted for use of an adult female or male user

At least one of the front panel 3 and the back panel 4, and preferably both the front panel 3 and the back panel 4 are formed from a stretch bonded laminate as produced in the continuous processes of the method for forming an array of pant-type articles as disclosed herein.

In FIG. 1b of the drawings a second example of a pant-type absorbent article 1, such as an article for an adult female or male user is schematically illustrated in an assembled and ready-to-use state. The difference between the absorbent articles in FIGS. 1a and 1b is that the absorbent article 1 in FIG. 1b has a unitary chassis 9″, i.e. the front and back panels 3, 4 comprises a web material which is continuous at least in the longitudinal direction Y of the pant-type absorbent article 1 and which includes a crotch portion 10 interconnecting the front and back panels 3, 4 and which is integrally formed with these regions. In the pant-type article in FIG. 1b, there is no clear delimitation between the front panel 3, the crotch portion 10 and the back panel 4.

The unitary chassis 9″ including the front panel 3, the back panel 4 and the interconnecting crotch portion 10 is fully or partly formed from a stretch bonded laminate as produced in the continuous processes of the method for forming an array of pant-type articles as disclosed herein.

At least one and preferably both of the front panel 3 and the back panel 4 in the example of FIG. 1a includes or consists of a stretchbonded elastic laminate as disclosed herein such as a laminate of an elastic film sandwiched between two fibrous outer layers. The front panel 3 extends in the longitudinal direction Y of the pant-type absorbent article 1 from a front leg edge 23 to a waist edge 21. In the example of FIG. 1a, the front panel 3 is provided with an elastic waist feature in the form of additional elastic elements 70 attached at a waist portion 42 of the pant-type absorbent article 1. The material in the back panel 4 is also preferably an elastic laminate as disclosed herein and may have the same structure as the front panel material.

The material in the front and back panels 3, 4 in the unitary chassis in the article in FIG. 1b may have the same structure as described with reference to FIG. 1a.

The shape of the front and back panels 3, 4 may be varied to fit a particular category of user. For example, one or both of the front panel and the back panels 3, 4 may have a substantially rectangular shape. Moreover, a leg edge region of the front and/or back panels 3, 4 may have a curved shape adapted to provide better conformance to a leg. As is illustrated in FIGS. 1a and 1b, the back panel 4 may have a greater extension in the longitudinal direction Y of the pant-type absorbent article than the front panel 3. However, the front and back panels 3,4 may have equal extensions in the longitudinal direction Y. Although generally less preferred, the front panel 3 may have a greater extension in the longitudinal direction Y of the pant-type absorbent article than the back panel 4.

As described herein, an objective with the present disclosure is to provide an array of absorbent articles in at least two sizes, for the purpose of providing absorbent articles with increased comfort and fit as well as discrete underwear-like visual appearance, while taking into account anatomical and size differences between different users, and while also maintaining a low manufacturing cost.

The method as disclosed herein is a method for producing an array of disposable pant-type articles comprising at least a first pant-type article and a second pant-type article having different size. The method comprises a number of different processes corresponding to the number of pant-type articles in the array, all processes involving forming a stretchbonded laminate web comprising a continuous elastic web and at least one non-elastic or less elastic web, forming a composite web of interconnected precursor articles comprising the stretchbonded laminate web and separating individual pant-type articles from the composite web by severing the composite web at side joins formed between the precursor articles.

In the different processes comprised in the method, the continuous elastic web differs between the different processes with regard to elastic properties while a pitch length of the stretchbonded elastic laminate web (31) is the same in all continuous processes of the method.

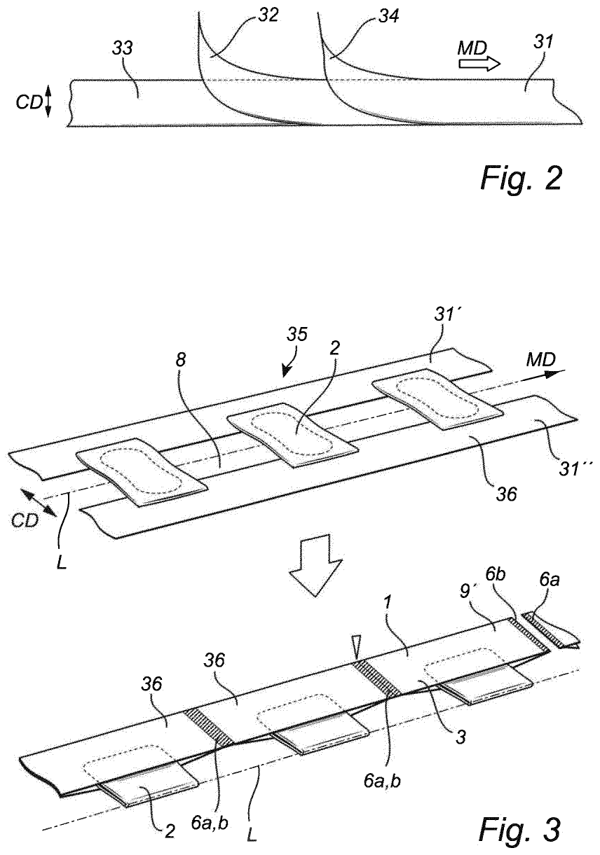

With reference to FIG. 2, there is shown a highly schematic representation of a process for manufacturing a stretchbonded laminate web 31 including providing a continuous elastic web 32 being elastically stretchable a machine direction, MD; moving the continuous elastic web 32 in the machine direction, MD; stretching the continuous elastic web 32 in the machine direction; and bonding the stretched continuous elastic web 32 while held in the stretched state to a non-elastic or less elastic web 33 to form the stretchbonded elastic laminate web 31. In FIG. 2 the stretchbonded elastic laminate web 31 is shown to include a further non-elastic or less elastic web 34, which is also stretchbonded to the continuous elastic web 32 such that the continuous elastic web 32 is sandwiched between the two non-elastic or less elastic webs 33, 34. The further non-elastic or less elastic web 34 is optional to the processes as disclosed herein. The non-elastic or less elastic web or webs 33, 34 may be fibrous nonwoven webs, and the continuous elastic web 32 may be an elastic film. The continuous elastic web 32 may be stretchable or elastically stretchable also in the cross machine direction CD, perpendicular to the machine direction, MD and the stretchbonding process may involve stretching of the continuous elastic web 32 in the cross machine direction CD.

The stretchbonded elastic laminate web may be composed of first and second outer layers of fibrous material and an elastic film located between the fibrous layers. The stretchbonded elastic laminate web may comprise one or more additional fibrous layers laminated to one or both of the first and second fibrous layers. Such additional fibrous layers may be present only in parts of the elastic laminate. Thus the elastic laminate need not be identical all over its area, but may comprise different layers in different areas.

It may be preferred that a continuous elastic web in the form of an elastic film is provided with fibrous nonwoven webs on both surfaces of the film. As disclosed herein, a stretchbonded nonwoven-film-nonwoven laminate web may alternatively be produced e.g. by stretchbonding a nonwoven web to an elastically stretchable bi-laminate of a nonwoven web and an elastic film which has been produced by other methods than stretchbonding, e.g. involving incremental stretching.

A stretchbonded elastic laminate web 31 having outer fibrous nonwoven layers is highly suitable for being arranged as an outside coversheet material as well as an inner coversheet material over at least part of the front panel, the back panel and the crotch portion of the chassis of the pant-type articles as disclosed herein. The stretchbonded elastic laminate web may constitute the sole component of the chassis in at least 20%, preferably at least 25%, more preferably at least 30% and most preferably at least 40% of the total surface area of the pant-type article, as seen in a flat state.

The elastic laminate may be absent in all or a substantial part of the crotch portion of the article. A crotch panel material may underlie at least part of the absorbent assembly on the garment-side thereof. The crotch panel material may be of a non-elastic web material, although elastic materials may also be used. Suitably, the crotch panel material is a nonwoven material. The crotch panel material is joined to the elastic laminate.

With reference to FIG. 3, manufacturing of a pant-type absorbent article 1 having a two-piece chassis 9′, e.g. as illustrated in FIG. 1a may be performed by manufacturing two parallel continuous strips of stretchbonded laminated elastic web 31′, 31″ forming the front and rear body panels 3, 4 of the finished absorbent article 1 and by intermittently connecting the continuous strips of stretchbonded laminated elastic web 31′, 31″ with a crotch material, such as a core insert 2.

Depending on the desired shape of the leg openings of the final pant-type article the web material may be cut and shaped to provide a more underwear-like appearance when assembled into a pant configuration.

The separately fabricated core insert 2 is placed in the gap between the strips 31′, 31″ of stretchbonded elastic laminate web while the strips 31′, 31″ are still in a tensioned state, such that the core insert 2 partly overlaps with the both strips 31′, 31″ of stretchbonded elastic laminate web. The absorbent insert 2 is secured to the strips 31′, 31″ by any suitable method, as set out herein.

In this manner a composite web 35 of article components is formed, the composite web 35 constituting a continuous web having leg openings 8 arranged therein and consisting of interconnected precursor articles 36. As set out herein, the composite web 35 comprises a stretchbonded elastic laminate web 31′, 31″ as a chassis component. As shown in the right-hand part of FIG. 3, the composite web 35 is folded along a fold line L, extending in the machine direction MD, centrally along the composite web 35.

After the composite web 35 has been folded, side joins are formed in the folded composite web 35 and individual pant-type articles 1 are separated from the composite web 35 by severing the composite web at the side joins 6a, 6b.

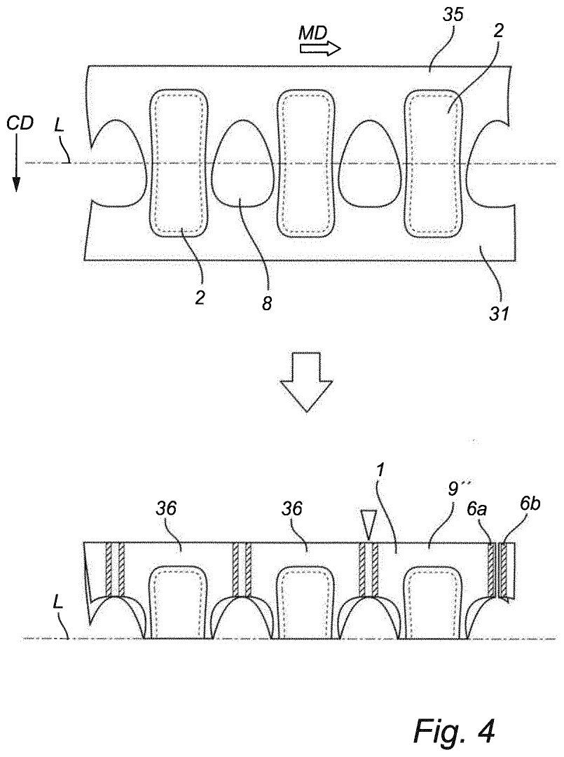

As illustrated by FIG. 3, the side joins may be produced as a single broad join 6a,b between the individual precursor articles 36, which join 6a,b is divided in two parts 6a, 6b when separating the finished pant-type articles 1 from the continuous web 35 of precursor articles 36. Alternatively, as illustrated in FIG. 4, two separate side joins 6a, 6b may be formed side by side between the individual precursor articles 36 whereafter the continuous web 35 of precursor articles 36 is severed between the two side joins 6a, 6b.

When the individual pant-type articles 1 have been separated from the composite web 35, the laminated elastic web of the front and back panels 3, 4 is no longer held in a stretched state. Thereby, the stretchbonded elastic web material in the front and back panels will cause the web material to gather, i.e. to contract and form rugosities or wrinkles in the non-elastic or less elastic web material which is bonded to the elastic web material.

The process which is schematically illustrated in FIG. 4, shows the production of pant-type absorbent articles having a unitary chassis. In FIG. 4, a composite web 35 of article components is formed by moving a stretchbonded elastic laminate web 31 in the machine direction MD; intermittently applying core inserts 2 to the moving stretchbonded elastic laminate web 31 and cutting out leg openings 8 between the core inserts 2. The composite web 35 of article components is subsequently folded along a fold line L extending in the machine direction, MD.

After the composite web 35 has been folded, side joins are formed in the folded composite web 35 and individual pant-type articles 1 are separated from the composite web 35 by severing the composite web at the side joins 6a, 6b as illustrated by the right-hand part of FIG. 4.

Although the process in FIG. 4 involves the use of a stretchbonded elastic laminate web 31 including an elastic web 32 being coextensive with the non-elastic or less elastic layer or layers 33, 34, it is to be understood that the elastic web material needs not be continuous in the cross machine direction, CD. By way of example, the elastic web may only be applied in one or both of the areas of the stretchbonded elastic laminate web 31 corresponding to the front and back panels 3,4 in the finished pant-type article. Furthermore, a process producing an absorbent pant-type article having a unitary chassis may be produced by applying absorbent cores, a barrier layer, an acquisition layer, leg elastic elements, etc. between a non-elastic or less elastic inner liner material and an outer elastic cover material during stretchbonding of the outer elastic cover material to the inner liner material.

The method as disclosed herein comprises the production of an array of pant-type articles, the array comprising at least two articles differing in size. In the method the differently sized articles in the array are produced by different processes, each process involving stretchbonding of a continuous elastic web to a non-elastic or less elastic web. In each different continuous process the continuous elastic web is different from the continuous elastic web in every other continuous process used for manufacturing the different size pant-type articles of the array. The continuous elastic webs of the different processes in the method differ from each other with regard to elastic properties although the pitch length of the stretchbonded elastic laminate web is the same in all the different continuous processes.

### DESCRIPTION OF TEST METHODS

### Elasticity Test

The method measures how an elastic material behaves at cycles of repeated load and unload. The sample is stretched to a predetermined elongation and a cyclic movement between 0 and said predetermined elongation is performed. Desired load and unload forces are recorded. The permanent, i.e. remaining, elongation of the relaxed material is measured.

A tensile tester, Lloyd LRX, able to perform cyclic movements and equipped with a printer/plotter or software presentation is used. The sample is prepared by cutting it to a width of 25 mm and a length that is preferably 20 mm longer than the distance between the clamps in the tensile tester.

The tensile tester is calibrated according to the apparatus instructions. The parameters needed for the test (load and unload forces) are adjusted to:
Crosshead speed: 500 mm/min
Clamp distance: 50 mm
Preload: 0.05 N

The sample is placed in the clamps according to the marks and it is made sure that the sample I centred and fastened perpendicularly in the clamps. The tensile tester is started and three cycles between 0 and the predetermined elongation equal to the highest defined 1^{st} load are performed. Before the last cycle, the sample is relaxed for 1 minute, then the permanent elongation is measured by stretching the sample until a force of 0.1 N is detected and the elongation is read.

An elastic material is defined as a material having a permanent elongation after relaxation of less than 10% after the material has been subjected to an elongation of 30% in the test above. An elongation of 30% means an elongation to a length that is 30% longer than the initial length of the sample.

A non-elastic material has a permanent elongation after relaxation of more than 10% after having been subjected to an elongation of 30%.

### Body Portion Circumference Test

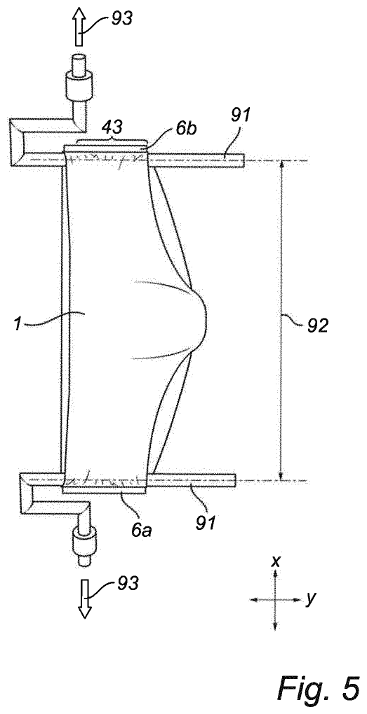

FIG. 5 shows schematically the test equipment used for measuring of the circumference of the body portion 43 in an extended state of a pant-type article 1.

Measurements of the circumference of the body portion 43 in an extended state of the pant-type article 1 may be performed by using any suitable tensile tester. The tensile tester that were used for obtaining the experimental data below was an Instron, model 4464, being equipped with parallel upper and lower tube-formed cylinders 91 having a diameter of 60 millimetres and being oriented in the longitudinal direction Y of the pant-type article 1. The tube-formed cylinders are made of blank polished SS2333 stainless steel with a smooth surface to provide low friction such that the pant-type articles do not stick to the surface of the tube-formed cylinders. The distance 92 between the cylinders 91 are initially set to correspond to the natural relaxed width of the pant-type article 1 such that the pant-type article can be mounted on the cylinders 91 in a non-extended state with the cylinders 91 placed inside the pant-type article 1 and with the cylinders 91 extending at least over the entire length of the side seams 6a, 6b. The cylinders 91 are subsequently pulled apart in the transverse direction X. The test is performed with a constant speed of 200 mm/minute up to 90 Newton tension force in which the tensile tester has been programmed to plot and/or sample data at least at 3, 10, 30 and 90 Newton.

For the measurements, new products were taken from a package and unfolded. The test result is based on an average result of 5 tested products. The distance 92 between the centres of the cylinders 91 was measured when applying a specified force in opposite directions 93. The circumference of the body portion 43 in an extended state of the absorbent article 1 corresponds to twice the distance 92 between the centre of the cylinders 91 plus the diameter 60 millimetres of the cylinders 91 multiplied with n. The circumference of the body portion 43 is measured at 3, 10, 30 and 90 Newton.

Comparison between the circumferences at different extension of the body portion of the pant-type articles in the array of pant-type articles should be made at 3 N, at 10 N, at 30 N and at 90 N in accordance with the described test method. Any waist elastic should be cut away before the measurements of the body portion circumference are carried out.

### Example

Two different pant-type articles, A and B, were produced and tested according to the body portion circumference test disclosed herein. The tested article A is suitable for use as a medium-size article and the tested article B is suitable for use as a small size article.

Both tested articles had the same construction with a chassis having a front panel and a back panel made from a stretchbonded laminate of an elastic film which had been stretchbonded between two spunbond nonwoven webs. The only difference between the tested articles was that the elastic film in test article A was Extretch MD which is a coextruded film with elastic polyolefin core and polyolefin skin layers provided by Exten S.A. and the elastic film in test article B was CEX-614 which is a coextruded film with elastic core comprising SBC and polyolefin elastomers and polyolefin skin layers, provided by Tredegar Corporation.

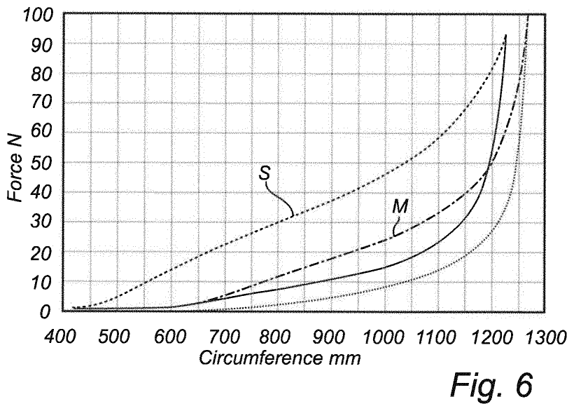

The results of the tests are shown in the diagram in FIG. 6, where the lower curve M shows the force/extension for test article A and the upper curve S shows the force/extension for test article B. As can be seen in FIG. 6, the circumference at 10 Newton of test article A is approximately 40% larger than the circumference of test article B at 10 Newton, the circumference at 30 Newton of test article A is approximately 35% larger than the circumference of test article B at 30 Newton, and the circumference of test article A is approximately 3% larger than the circumference of test article B at 90 Newton.

1. An array of pant-type articles, such as an array of absorbent pant-type articles including an absorbent core, said array of pant-type articles including a first pant-type article and a second pant-type article, each said first pant-type article and said second pant-type article comprising a chassis, said chassis comprising a front panel, a back panel and a crotch portion between said front and back panels, said front panel having a waist edge and a pair of side edges, and said back panel having a waist edge and a pair of side edges, said side edges of said front and back panels being joined in a first and a second side seam, whereby said pant-type article has a waist opening and a first and a second leg opening,
wherein each said first pant-type article and said second pant-type article comprises a stretchbonded elastic laminate web comprising an elastic web and an in-elastic or less elastic web, said elastic web of said first pant-type article differing from said elastic web of said second pant-type article in at least one or more aspects selected from:
physical structure;
polymer composition;
basis weight;
thickness;
permanent set;
degree of orientation in a machine direction of said elastic web, said stretchbonded elastic laminate web extending from said first side seam to said second side seam over at least a part of said front panel and/or of said back panel of said chassis,
wherein:
a body portion circumference at 10 Newton of said first pant-type article in said array is at least 20% larger than a body portion circumference of said second pant-type article in said array at 10 Newton,
a body portion circumference at 30 Newton of said first pant-type article in said array is at least 20% larger than a body portion circumference of said second pant-type article in said array at 30 Newton, and
a body portion circumference at 90 Newton of said first pant-type article in said array is less than 10% larger than a body portion circumference of said second pant-type article in said array at 90 Newton as measured by the body portion circumference test as disclosed herein.

2. The array of pant-type articles according to claim 1, wherein said stretchbonded elastic laminate web constitutes a sole component of said chassis in in 20%-100% of a total surface area of said pant-type article, as seen in a flat state.

3. The array of pant-type articles according to claim 1, wherein each said first pant-type article and said second pant-type article comprises a two-piece chassis.

4. The array of pant-type articles according to claim 1, wherein each said first pant-type article and said second pant-type article comprises a unitary chassis.

5. A method for producing pant-type articles for an array of pant-type articles, said array of pant-type articles comprising a first pant-type article and a second pant-type article, said first and second pant-type articles differing in size, wherein said method comprises a first continuous process for producing said first pant-type article and a second continuous process for producing said second pant-type article, each said first and second continuous process including:
providing a continuous elastic web and moving said continuous elastic web in a machine direction, said continuous elastic web being elastically stretchable in said machine direction;
stretching said continuous elastic web in said machine direction;
bonding said stretched continuous elastic web while held in said stretched state to a non-elastic or less elastic web to form a stretchbonded elastic laminate web;
forming a composite web of article components, said composite web constituting a continuous web having leg openings arranged therein and consisting of interconnected precursor articles, said composite web comprising said stretchbonded elastic laminate web as a chassis component;
folding said composite web along a fold line extending along said composite web in said machine direction;
forming side joins in said folded composite web; and
separating individual pant-type articles from said composite web by severing said composite web at said side joins,
wherein said continuous elastic web in said first continuous process is different from said continuous elastic web in said second continuous process in one or more aspects selected from:
physical structure
polymer composition;
basis weight;
thickness;
permanent set;
degree of orientation in said machine direction of said elastic web; and
wherein a pitch length of said stretchbonded elastic laminate web is the same in said first continuous process and said second continuous process.

6. The method according to claim 5, wherein forming said composite web of article components comprises:
moving said stretchbonded elastic laminate web as a first continuous web in said machine direction alongside a second continuous web arranged at a distance from said first continuous web in a cross machine direction perpendicular to said machine direction with a gap between said first continuous web and said second continuous web;
intermittently connecting said first continuous web with said second continuous web by attaching a crotch material to said first continuous web and to said second continuous web, said crotch material bridging said gap between said first continuous web and said second continuous web, wherein said second continuous web is a non-elastic or elastic web.

7. The method according to claim 5, wherein forming said composite web of article components comprises:
moving said stretchbonded elastic laminate web in said machine direction;
intermittently cutting out leg openings in said stretchbonded elastic laminate web.

8. The method according to claim 5, wherein said elastic web is stretched in said machine direction by at least 90% of an initial non-stretched length during stretch-bonding.

9. The method according to claim 5, wherein said stretched elastic web is bonded to said non-elastic or less elastic web by means of adhesive that is applied to one or both webs by means of coating, spraying, extrusion or meltblowing, or that said stretched elastic web is bonded to said non-elastic or less elastic web by means of ultrasonic welding or thermobonding.

10. The method according to claim 5, wherein said article components include one or more of:
an absorbent core or core insert;
a waist elastic member;
a leg elastic member;
an inner liner;
an acquisition layer;
a barrier layer;
a barrier cuff; and
a fastener element.

11. The method according to claim 5, wherein said continuous elastic web comprises an elastic film, and/or wherein said non-elastic or less elastic web is a nonwoven web.

12. The method according to claim 11, wherein said continuous elastic web is a laminate comprising an elastic film and a nonwoven web.

13. An array of pant-type articles, such as an array of absorbent pant-type articles including an absorbent core, said array of pant-type articles including a first pant-type article and a second pant-type article, wherein said array of pant-type articles is produced according to the method of claim 5 and wherein said first pant-type article differs in size from that of said second pant-type article.

14. The array of pant-type articles according to claim 13, wherein:
a body portion circumference at 10 Newton of said first pant-type article in said array is at least 20% larger than a body portion circumference of said second pant-type article in said array at 10 Newton,
a body portion circumference at 30 Newton of said first pant-type article in said array is at least 20% larger than a body portion circumference of said second pant-type article in said array at 30 Newton, and
a body portion circumference at 90 Newton of said first pant-type article in said array is less than 10% larger than a body portion circumference of said second pant-type article in said array at 90 Newton as measured by the body portion circumference test as disclosed herein.

15. The array of pant-type articles according to claim 1, wherein a body portion circumference at 90 Newton of said first pant-type article in said array is less than 5% larger than a body portion circumference of said second pant-type article in said array at 90 Newton as measured by the body portion circumference test as disclosed herein.

16. The array of pant-type articles according to claim 1, wherein said stretchbonded elastic laminate web constitutes a sole component of said chassis in in 40%-100% of a total surface area of said pant-type article, as seen in a flat state.

17. The array of pant-type articles according to claim 1, wherein the first pants-type article and the second pants-type article are each produced with a pitch length of said stretchbonded elastic laminate web that is the same.

18. The method according to claim 5, wherein said elastic web is stretched in said machine direction by at least 300% of an initial non-stretched length during stretch-bonding.

19. The method according to claim 5, wherein said continuous elastic web consists of a breathable elastic film, and wherein said non-elastic or less elastic web is a nonwoven web.
